(19) United States
(12) Patent Application Publication
(10) Pub. No.: US 2026/0284130 A1
(43) Pub. Date: Sep. 24, 2026

(54) COMPOSITION FOR PREVENTING, AMELIORATING OR TREATING EYE DISEASES

(71) Applicant: JULIA LABORATORY, Suwon-si, Gyeonggi-do (KR)

(72) Inventors: Wan Jin JAHNG, Suwon-si (KR); Kyunghae LEE, Suwon-si (KR); Jin Sook KIM, Seoul (KR)

(73) Assignee: JULIA LABORATORY, Suwon-si, Gyeonggi-do (KR)

(21) Appl. No.: 19/476,008

(22) PCT Filed: Apr. 18, 2024
(86) PCT No.: PCT/KR2024/005246

§ 371 Date: Dec. 5, 2025

(30) Foreign Application Priority Data
Apr. 18, 2023 (KR) 10-2023-0050603

(51) Int. Cl. A61K 36/21; A61K 9/00; A61K 36/484; A61P 27/02

(52) U.S. Cl. CPC A61K 36/21; A61K 9/0056; A61K 9/0095; A61K 36/484; A61P 27/02; A61K 2236/333

(57) ABSTRACT

The present invention relates to a composition for preventing, ameliorating or trating eye diseases, comprising Achyranthes japonica, Glycyrrhiza uralensis, or a mixed extract thereof. The composition comprising Achyranthes japonica, Glycyrrhiza uralensis, or a mixed extract thereof as an active ingredient, according to the present invention, has an excellent antioxidant effect and an excellent effect of restoring oxygen consumption and ATP production in mitochondria, and further has the effects of protecting mitochondria and inhibiting retinal cell death caused by blue light stress, and thus can be applied as a medicine, a health functional food, and a quasi-drug composition, for the prevention, amelioration or treatment of various eye diseases.

### TECHNICAL FIELD

The present invention relates to a composition for preventing or treating eye diseases, and more specifically, to a composition for preventing, ameliorating or treating eye diseases including Achyranthes japonica, Glycyrrhiza uralensis, or a mixed extract thereof.

### BACKGROUND ART

Age-related macular degeneration (AMID), which is one of the world's top three eye diseases, is classified as dry and wet types. In 2020, the total number of AMD patients reached 196 million. Among them, 20 to 30% of dry AMD patients progress into wet AMD, which causes blindness following rapid vision decline. The dry AMD rapidly propagates in all age groups due to environmental changes such as population aging, frequent use of smart phones, etc. A degenerative disease progresses from vision deterioration to blindness due to the death of photoreceptors as the waste, drusen, accumulates on the macula due to degeneration of retinal cell functions, and reportedly accounts for 90% of macular degeneration. It is reported that the dry AMD outbreak relates to complex and diverse mechanisms such as oxidative stress, inflammation, decreased mitochondrial function, abnormal angiogenesis, etc.

In particular, mitochondria plays an important role in energy production, reactive oxygen, cell death, and retrograde signaling information, and retrograde signaling in mitochondria regulates complement, inflammation, and innate immune pathways involved in the progression of macular degeneration, and thus the severity of AMD depends on the degree of mitochondrial DNA damage and fragmentation of retinal pigmented epithelial cells. In addition, mitochondrial permeability may be regulated to delay and regulate an AMD outbreak and a progression rate thereof through the utilization of a substrate or a regulator for preventing cell death and the suppression of oxidative stress (ROS) damage which affects mitochondrial functions.

It is reported that protecting the retinal mitochondria responsible for these various functions may prevent, delay, or treat the development of macular degeneration (J Ophthalmic Vis Res 2017; 12 (4): 424-8).

In the meantime, there has been no therapeutic agent to restore vision in most dry AMD patients, but in February 2023, the United States Food and Drug Administration (FDA) recently approved Syfovre (Apellis Pharmaceuticals Inc., USA) as the first new drug for geographic atrophy (GA) patients due to late dry macular degeneration. It was announced that the new drug inhibits the excessive activity of complement 3 involved in an immune chain reaction, is injected at intervals of 25 to 60 days, and would be released at $2,190 (about 2.8 million KRW) per vial from March 23 (PHARM EDAILY; Feb. 21, 2023). However, it is expected that the new drug will have a very large limit to be applied to most patients with very expensive treatment costs.

In addition, “Zimura (Iveric Bio Inc., USA)” is a drug currently undergoing phase III clinical trials, and its mechanism is to prevent the decomposition of C5 among complement factors into C5a and C5b, thus killing chronic inflammation or cells. Furthermore, US Allegro and Hanmi Pharmaceutical are preparing for the phase III of “Luminate” as a candidate for new drugs for dry AMD patients, and unlike Syfovre, which targets late dry AMD, Luminate is reported as an early treatment strategy.

However, most of the therapeutic agents, which have been currently approved or under development, are vitreous injections, and there is a problem in that there is no product which may be administered in combination with a therapeutic agent currently used as an oral administration agent.

Meanwhile, Glycyrrhiza uralensis (Glycyrrhizae glabra; Glycyrrhizae radlix et rhizoma) is a perennial grass belonging to the legume, and its roots are dried and used as a medicinal ingredient. In general, Glycyrrhiza uralensis is used to treat inflammation, gastritis, stomatitis, sore throat, hepatitis, eczema, peptic ulcers, alcohol detoxification, etc.

Achyranthes japonica (Achyranthes bidentata; Achyranthes radix) belongs to the Amaranthaceae family, protects the liver and kidneys, and dissolves the blood stasis caused by blood circulation disorders. In addition, it has muscular and skeletal enhancement effects, ameliorates arthritis, and is used to treat menstrual irregularity, contusion, and backache, but has oxytocic activity, thus reportedly suggesting that pregnant women need to be careful about taking it.

### DISCLOSURE

### Technical Problem

The present inventors have confirmed that Achyranthes japonica, Glycyrrhiza uralensis, or a mixed extract thereof has an excellent antioxidant effect and an excellent effect of restoring oxygen consumption and ATP production in mitochondria, and further has the effects of protecting mitochondria and inhibiting retinal cell death caused by blue light oxidative stress, thereby completing the present invention.

### Technical Solution

One object of the present invention is to provide a food composition for preventing or ameliorating eye diseases, including Achyranthes japonica, Glycyrrhiza uralensis, or a mixed extract thereof as an active ingredient.

Another object of the present invention is to provide a pharmaceutical composition for preventing or treating eye diseases, including Achyranthes japonica, Glycyrrhiza uralensis, or a mixed extract thereof as an active ingredient.

Still another object of the present invention is to provide a quasi-drug composition for preventing or ameliorating eye diseases, including Achyranthes japonica, Glycyrrhiza uralensis, or a mixed extract thereof as an active ingredient.

Still another object of the present invention is to provide a feed composition for preventing or ameliorating eye diseases, including Achyranthes japonica, Glycyrrhiza uralensis, or a mixed extract thereof as an active ingredient.

Still another object of the present invention is to provide a method for preventing or treating eye diseases, including administering the pharmaceutical extract into an individual.

Still another object of the present invention is to provide a use of the composition including Achyranthes japonica, Glycyrrhiza uralensis, or a mixed extract thereof as an active ingredient for preventing or treating eye diseases.

### Advantageous Effects

In one aspect, the composition of the present invention, including Achyranthes japonica, Glycyrrhiza uralensis, or a mixed extract thereof as an active ingredient, can have an excellent antioxidant effect in retinal epithelial cells and an excellent effect of restoring oxygen consumption and ATP production in mitochondria, and can further have the remarkably excellent effects of protecting mitochondria and inhibiting retinal cell death caused by blue light oxidative stress, and thus can be utilized as a medicine, a health functional food, and a quasi-drug composition, for the prevention, amelioration or treatment of eye diseases. In addition, there can be advantages in that it needs a smaller number of medicinal ingredients essentially included therein and has a less drug interaction, thus providing excellent commercial viability and a row risk of side effects.

### DESCRIPTION OF DRAWINGS

FIG. 1 is a graph showing the cell viability of each extract from Achyranthes japonica, Glycyrrhiza uralensis, or a mixed extract thereof according to one embodiment in a retinal epithelial cell line ARPE-19.

FIG. 2 is a graph showing the cell protective efficacy of an extract (200 μg/ml) from Achyranthes japonica, Glycyrrhiza uralensis, or a mixed extract thereof according to one embodiment in ARPFE-19 cells treated with tBHP (300 uM).

FIG. 3 is a graph showing the cell protective efficacy of an extract (1 to 200 μg/ml) from Achyranthes japonica, Glycyrrhiza uralensis, or a mixed extract thereof according to one embodiment in ARPE-19 cells treated with tBHP (300 uM).

FIG. 4 is a graph showing the efficacy of JK-502, 504, 505, 508, 509, 514, and 520 from Achyranthes japonica, Glycyrrhiza uralensis, or a mixed extract thereof according to one embodiment in the tBHP-treated ARPE-19 with regard to the mitochondrial respiratory system (Oligo (Oligomycin, ATP synthesis inhibitor), FCCP (fluoro-carbonyl cyanide phenylhydrazone (FCCP), hydrogen ions transporter), and rotenone/antimycin A (ROT/AA); complex I and III inhibitors)).

FIG. 5 is a graph showing the effect of JK-504, 505, 508, 509, 514, and 520 from Achyranthes japonica, Glycyrrhiza uralensis, or a mixed extract thereof according to one embodiment in the tBHP-treated ARPE-19 with regard to protecting mitochondria.

FIGS. 6 to 9 are graphs showing the effect of Achyranthes japonica, Glycyrrhiza uralensis, or a mixed extract thereof according to one embodiment on inhibiting retinal cell death caused by blue light oxidative stress.

### BEST MODE

The present invention will be described below in detail. Meanwhile, the description and embodiments disclosed in the present invention may also be applied to other descriptions and embodiments. In other words, all the combinations of various elements disclosed in the present invention may fall within the scope of the present invention. In addition, the scope of the present invention is not limited to the specific description described below.

Furthermore, those skilled in the art may recognize or confirm a number of equivalents to the specific aspects of the present invention described herein through conventional experiments. It is also intended that such equivalents are included in the present invention.

Hereinafter, the present invention will be described in more detail.

The present invention may be based on the new concept that Achyrauthes japonica, Glycyrrhiza uralensis, or a mixed extract thereof has an excellent antioxidant effect and an excellent effect of restoring oxygen consumption and ATP production in mitochondria, and further has the effects of protecting mitochondria and inhibiting retinal cell death caused by blue light oxidative stress.

In particular, the present invention may be based on the new confirmation that Achyranthes japonica, Glycyrrhiza uralensis, or a mixed extract thereof has a remarkably excellent antioxidant effect in retinal epithelial cells and an excellent effect of restoring oxygen consumption and ATP production in mitochondria, and further has the effects of protecting mitochondria and inhibiting retinal cell death caused by blue light oxidative stress at a specific mixing ratio when an extraction solvent is 30% to 50% ethanol.

To achieve the above objects, one aspect of the present invention may provide a food composition for preventing or ameliorating eye diseases, including Achyranthes japonica, Glycyrrhiza uralensis, or a mixed extract thereof as an active ingredient.

In the present specification, the scientific term “Achyranthes japonica; Achyranthes bidentata; Achyranthis radix” may belong to the Anaranthaceae family, and may reportedly protect the liver and kidneys, dissolve the blood stasis caused by blood circulation disorders, have muscular and skeletal enhancement effects, ameliorate arthritis, and is used to treat menstrual irregularity, contusion, and backache, but may have oxytocic activity, thus reportedly suggesting that pregnant women need to be careful about taking it.

Achyrnthes japonica used as a herbal medicine material may be any one or more selected from the group consisting of roots, root stems, leaves, flowers, seeds, and whole plants.

In the present specification, the scientific term Glycyrrhizae uralensis; Glycyrrhizae glabra; Glycyrrhizae radix et rhizoma may be a perennial grass belonging to the legume, and its roots may be dried and used as a medicinal ingredient. In general, Glycyrrhiza uralensis may be reportedly used to treat inflammation, gastritis, stomatitis, sore throat, hepatitis, eczema, peptic ulcers, alcohol detoxification, etc.

In the present specification, Glycyrrhiza uralensis used as a herbal medicine material of the extract may be any one or more selected from the group consisting of above-ground parts such as flowers, leaves, stems, etc., roots, seeds, and whole plants.

Above Achyranthes japonica and/or Glycyrrhiza uralensis used herein may include one which is commercially purchased or may be harvested from nature or grown without limitation.

In the present specification, the term “extract” may refer to a product such as a liquid component obtained by immersing a desired material in various solvents and then extracting from the same at room temperature or in a heated state for a certain period of time, a solid content obtained by removing the solvent from the liquid component, and the like. Furthermore, in addition to the product, it may be comprehensively construed to include all of a diluent of the product, a concentrate thereof, a crude purified product thereof, a purified product thereof, etc. Accordingly, the Achyranthes japonica, the Glycyrrhiza uralensis, or the mixed extract thereof provided in the present invention may be construed to include an extraction liquid per se and extracts of all formulations which may be formed using the extraction liquid, such as each resulting extraction liquid or a mixed solution thereof, a mixed solution obtained by mixing and extracting, a diluent or a concentrated solution of the extraction liquid, a dried product obtained by drying the extraction liquid, a crude purified product or a purified product of the extraction liquid, a mixture thereof, or the like.

In addition, the Achyranthes japonica, the Glycyrrhiza uralensis, or the mixed extract thereof may be extracted from various organs of a natural plant, a hybrid plant, and a mutant plant, and specifically may be extracted from a plant tissue culture as well as from a root, an above-ground part, a stem, a leaf, a flower, a trunk of a fruit, and a bark of the fruit.

Furthermore, the extract may be based on a concept including any active ingredient separated from natural products (herbal medicines) through an appropriate solvent without limitation on the method of separating or the type of the ingredient. In other words, it may be broadly defined to include all of a product obtained by extracting a component dissolved in a solvent from a natural product using water or an organic solvent, a product obtained by extracting only a specific component of a natural product, for example, a specific component such as oil, and the like, and may be used to encompass all of an extract and/or a purified product obtained in each step of extraction and/or purification, a diluent and/or a concentrated liquid of the extract and/or the purified product, a dry product of the extract, the purified product, the diluent, and/or the concentrated liquid, and the like.

The mixed extract of Achyranthes japonica and Glycyrrhiza uralensis, which is an active ingredient of the composition provided in the present specification, may refer to a mixture of an Achyranthes japonica extract and a Glycyrrhiza uralensis extract (a mixture of herbal medicine extracts), or may be a mixed extract obtained through extraction after mixing Achyranthes japonica and Glycyrrhiza uralensis.

The Achyranthes japonica extract, the Glycyrrhiza uralensis extract, and/or the extract thereof may be a crude extract, a solvent (polar solvent or non-polar solvent) soluble extract, a fraction, or a combination thereof, which are obtained by being extracted, separated, and/or fractionated from natural products using extraction, separation, and/or fractionation methods known in the art. In one specific embodiment, the Achyranthes japonica extract, the Glycyrrhiza uralensis extract, and/or the extract of mixtures thereof may be a crude extract or a solvent-soluble extract (e.g., a polar solvent-soluble extract), but is not limited thereto.

The Achyranthes japonica, the Glycyrrhiza uralensis, or the mixed extract thereof may be obtained by extracting from Achyranthes japonica, Glycyrrhiza uralensis, or the mixed extract thereof using at least one selected from water, an organic solvent, and a combination thereof as an extraction solvent. The organic solvent may be at least one selected from straight or branched-chain alcohols having one to four carbon atoms (specifically, methanol, ethanol, propanol, isopropanol, butanol, isobutanol, etc.), specifically may be water-soluble ethanol, and more specifically may be a 30% to 50% (v/v) aqueous ethanol solution.

In one specific embodiment, the extraction solvent may be water, ethanol, methanol, or a combination thereof (specifically, an aqueous ethanol solution, an aqueous methanol solution, and/or the like), and specifically 10 to 80% (v/v), 15 to 80% (v/v), 20 to 80% (v/v), 25 to 80% (v/v), 28 to 80% (v/v), 30 to 80% (v/v), 10 to 70% (v/v), 15 to 70% (v/v), 20 to 70% (v/v), 25 to 70% (v/v), 28 to 70% (v/v), 30 to 70% (v/v), 10 to 60% (v/v), 15 to 60% (v/v), 20 to 60% (v/v), 25 to 60% (v/v), 28 to 60% (v/v), 30 to 60% (v/v), 10 to 50% (v/v), 15 to 50% (v/v), 20 to 50% (v/v), 25 to 50% (v/v), 28 to 50% (v/v), or 30 to 50% (v/v) ethanol or methanol, specifically a 25 to 55% aqueous ethanol solution, and more specifically 30% to 50% (v/v) aqueous ethanol solution.

In one embodiment, the Achyranthes japonica, the Glycyrrhiza uralensis, or the mixed extract thereof may be
(1) a mixture between (i) an extract of Achyranthes japonica obtained by extracting from Achyranthes japonica (e.g., roots) with 10 to 80% (v/v), 15 to 80% (v/v), 20 to 80% (v/v), 25 to 80% (v/v), 28 to 80% (v/v), 30 to 80% (v/v), 10 to 70% (v/v), 15 to 70% (v/v), 20 to 70% (v/v), 25 to 70% (v/v), 28 to 70% (v/v), 30 to 70% (v/v), 10 to 60% (v/v), 15 to 60% (v/v), 20 to 60% (v/v), 25 to 60% (v/v), 28 to 60% (v/v), 30 to 60% (v/v), 10 to 50% (v/v), 15 to 50% (v/v), 20 to 50% (v/v), 25 to 50% (v/v), 28 to 50% (v/v), or 30 to 50% (v/v) ethanol or methanol and (ii) an extract of Glycyrrhiza uralensis obtained by extracting from Glycyrrhiza uralensis (e.g., above-ground parts) with 10 to 80% (v/v), 15 to 80% (v/v), 20 to 80% (v/v), 25 to 80% (v/v), 28 to 80% (v/v), 30 to 80% (v/v), 10 to 70% (v/v), 15 to 70% (v/v), 20 to 70% (v/v), 25 to 70% (v/v), 28 to 70% (v/v), 30 to 70% (v/v), 10 to 60% (v/v), 15 to 60% (v/v), 20 to 60% (v/v), 25 to 60% (v/v), 28 to 60% (v/v), 30 to 60% (v/v), 10 to 50% (v/v), 15 to 50% (v/v), 20 to 50% (v/v), 25 to 50% (v/v), 28 to 50% (v/v), or 30 to 50% (v/v) ethanol or methanol;
(2) an extract of mixtures obtained by extracting from Achyranthes japonica (e.g., roots) and Glycyrrhiza uralensis (e.g., above-ground parts) with 10 to 80% (v/v), 15 to 80% (v/v), 20 to 80% (v/v), 25 to 80% (v/v), 28 to 80% (v/v), 30 to 80% (v/v), 10 to 70% (v/v), 15 to 70% (v/v), 20 to 70% (v/v), 25 to 70% (v/v), 28 to 70% (v/v), 30 to 70% (v/v), 10 to 60% (v/v), 15 to 60% (v/v), 20 to 60% (v/v), 25 to 60% (v/v), 28 to 60% (v/v), 30 to 60% (v/v), 10 to 50% (v/v), 15 to 50% (v/v), 20 to 50% (v/v), 25 to 50% (v/v), 28 to 50% (v/v), or 30 to 50% (v/v) ethanol or methanol;
or (3) a combination thereof, and more specifically may be an extract of Achyranthes japonica, an extract of Glycyrrhiza uralensis, a mixed solution thereof, or an extracted liquid obtained after mixing and extracting, which is obtained by extracting with 30 to 50% (v/v) aqueous ethanol solution.

The extraction may be performed at an extraction temperature of 30 to 100° C., 40 to 100° C., 50 to 100° C., 60 to 100° C., 30 to 95° C., 40 to 95° C., 50 to 95° C., 30 to 90° C., 40 to 90° C., 50 to 90° C., 30 to 85° C., 40 to 85° C., 50 to 85° C., 40 to 70° C., or 50 to 70° C., but is not limited thereto.

The extraction may be performed under the above-described extraction solvent and/or extraction temperature conditions for 10 minutes or longer, 30 minutes or longer, one hour or longer, two hours or longer, or three hours or longer, for example, 10 minutes to 12 hours, 10 minutes to six hours, 10 minutes to five hours, 10 minutes to four hours, 10 minutes to three hours, 30 minutes to 12 hours, 30 minutes to six hours, 30 minutes to five hours, 30 minutes to four hours, 30 minutes to three hours, one to 12 hours, one to six hours, one to five hours, one to four hours, one to three hours, two to 12 hours, two to six hours, two to five hours, two to four hours, two to three hours, three to 12 hours, three to six hours, three to five hours, or three to four hours, but is not limited thereto.

The extraction may be performed by all commonly used extraction methods, and specifically may be performed by at least one method selected from the group consisting of hot water extraction, ultrasonic extraction, reflux extraction, cold extraction, reflux cooling extraction, solvent extraction, steam distillation, elution, compression, etc., and specifically may be reflux extraction, but is not limited thereto.

After the extract is obtained using the extraction solvent as described above, a liquid product may be obtained by cold-dipping, heating and/or filtering at room temperature through a conventional method known in the art, and the solvent may be further evaporated, spray-dried, or freeze-dried by an optional process. In addition, the extract obtained by extracting as described above may be concentrated under reduced pressure to be prepared in a powder state through additional processes such as freeze drying, vacuum distillation, and freeze drying, spray drying or the like.

The Achyranthes japonica extract, the Glycyrrhiza uralensis extract, and/or the mixed extract thereof may be obtained as an additionally purified fraction by using various chromatography techniques such as silica gel column chromatography, thin layer chromatography, high performance liquid chromatography, etc.

A mixing ratio of Achyranthes japonica extract and Glycyrrhiza uralensis extract in the mixed extract or a mixing ratio of Achyranthes japonica and Glycyrrhiza uralensis in the mixture of Achyranthes japonica and Glycyrrhiza uralensis may be 9:1 to 1:9, 3:1 to 1:9, 2:1 to 1:9, 1:1 to 1:9, 1:3 to 1:9, 1:6 to 1:9 (here, the weight of Achyranthes japonica or the solid content weight of Achyranthes japonica extract: the weight of Glycyrrhiza uralensis or the solid content weight of Glycyrrhiza uralensis extract) on a weight basis, and the content of Glycyrrhiza uralensis extract or Glycyrrhiza uralensis may be twice or more, three times or more, four times or more, five times or more, six times or more, seven times or more, eight times or more, or nine times or more (specifically 1:1 to 1:9, 1:1 to 1:8, 1:1 to 1:7, 1:1 to 1:6, 1:1 to 1:5, 1:1 to 1:4, 1:1 to 1:3, 1:1 to 1:2 (the weight of Achyranthes japonica or the solid content weight of Achyranthes japonica extract: the weight of Glycyrrhiza uralensis or the solid content weight of Glycyrrhiza uralensis extract)) than the content of Achyranthes japonica extract or Achyranthes japonica on a weight basis, and more specifically Achyranthes japonica:Glycyrrhiza uralensis may be a mixing (weight) ratio of 1:3 to 1:9, and more specifically may be a mixing ratio of 1:6 to 1:9.

The solid content weight may mean the weight of the solid remaining after removing a solvent component from the extract. This may be a term used to indicate that when the mixture is a mixture of Achyranthes japonica extract and Glycyrrhiza uralensis extract, it means a ratio between the weights of the active ingredients from which the extraction solvent has been removed so that the mixing ratio is not affected by the properties and/or concentration of the extract.

The Achyranthes japonica, the Glycyrrhiza uralensis, or the mixed extract thereof included as an active ingredient in the food composition may be in the form of a dried product, a concentrated product, or a concentrated dried product.

In addition, a known extract of a natural substance or a known component thereof, which enhances the effect of preventing or ameliorating eye diseases, may be selectively added to the extract of the present invention.

In the present specification, the term “eye disease” may refer to a disease occurring in an eye, and specifically may be macular degeneration, glaucoma, diabetic retinopathy, retinal edema, an epiretinal membrane, a retinal break, cataract, dry eye, superficial keratitis, or retinal and corneal abnormalities of unspecified cause (unknown cause), but is not limited thereto.

As used herein, the term “macular degeneration” may refer to age-related macular degeneration (AMD), which is one of the irreversible diseases and is a representative ophthalmic disease that develops in many seniors and induces blindness. Worldwide, the incidence is gradually increasing due to population aging. In the United States, macular degeneration may be a main cause of blindness, and environmental and genetic factors may also affect the outbreak. In particular, smoking has been reported to be the most deadly cause. In addition, obesity and consumption of excessive antioxidants and dietary fat may also cause macular degeneration and have an effect of further aggravating the same. Thus, healthy diet, weight control, proper exercise, no smoking, etc., may lower the outbreak of macular degeneration.

As used herein, the term “dry eye syndrome” may be a disease caused by inflammation of the lacrimal gland and denervation of the cornea to suppress tear secretion, abnormal tear evaporation due to a meibomian gland dysfunction or an eyelid dysfunction, or the like, and may be a corneal conjunctival epithelial disorder, in which tears are excessively dried due to the dry eye syndrome and appear as a corneal wound.

As used herein, the term “glaucoma” may be a progressive optic neuropathy which causes abnormality in the function of the optic nerve and generates a defect in the corresponding field of vision. The optic nerve may be a nerve which transmits the light received by the eye to the brain to “see,” and thus if a disorder occurs here, the visual field defect may appear, and the vision may be lost at the end. An open-angle glaucoma may refer to a glaucoma which occurs while an anterior chamber angle is not closed and is maintained in a normal form, and a closed-angle glaucoma may refer to a glaucoma which occurs when the anterior chamber angle is closed as the iris moves toward the cornea due to a sudden rise in back pressure. The angle formed by a rear surface of the cornea and a front surface of the iris may be called the anterior chamber angle, and when this is pressed, a passage through which aqueous humor is discharged may be blocked, and thus an intraocular pressure may rapidly rise.

As used herein, the term “retinopathy” may be called retinopathies and may refer to causing continuous or severe damage to the retina of the eye.

As used herein, the term “retinal edema” may be also called macular edema and may refer to edema in the macula, which is a central part of the retina. Seeing things may be performed by forming an image on the retina and delivering the same to the brain through the optic nerve, and the macula may be located in the middle of the retina and may be in charge of central vision. Thus, when macular edema is accompanied and abnormality occurs in the structure of the macula region, it may cause an abnormality in vision where objects appear to be dented or vision is reduced.

In the present specification, a food composition may be meant to include a health functional food, and the food composition and the health functional food may be interchangeable with each other in an equivalent sense, unless otherwise specified.

The “health functional food” may be a food prepared (processed) using nutrients which are likely to be deficient in daily meals or raw materials or ingredients with useful functions for the human body (hereinafter, referred to as “functional raw materials”), and may refer to all foods which help maintain health or prevent and/or ameliorate certain diseases or symptoms, and there is no particular limitation on a final product form. For example, the health functional food may be selected from the group consisting of various foods, beverages, food additives, and the like, but is not limited thereto.

The food composition may include an acceptable food supplementary additive, and may further include an appropriate carrier, excipient, and diluent commonly used in the preparation of health functional foods.

When the food composition is used as a food additive, the composition may be added as it is or used together with other foods or food ingredients, and may be appropriately used according to a conventional method.

The content of the active ingredient (Achyranthes japonica, Glycyrrhiza uralensis, or a mixed extract thereof) contained in the food composition may be appropriately determined without particular limitation according to the shape, desired effect, purpose, and the like of the food, and may be 0.001 to 99 wt %, 0.001 to 90 wt %, 0.01 to 99 wt %, 0.01 to 95 wt %, 0.01 to 90 wt %, 0.01 to 80 wt %, 0.01 to 50 wt %, 0.1 to 99 wt %, 0.1 to 95 wt %, 0.1 to 90 wt %, 0.1 to 80 wt %, 0.1 to 70 wt %, 0.1 to 60 wt %, 0.1 to 50 wt %, 0.1 to 30 wt %, 0.1 to 10 wt %, 1 to 99 wt %, 1 to 95 wt %, 1 to 90 wt %, 1 to 80 wt %, 1 to 70 wt %, 1 to 60 wt %, 1 to 50 wt %, 1 to 30 wt %, 1 to 10 wt %, 10 to 99 wt %, 10 to 95 wt %, 10 to 90 wt %, 10 to 80 wt %, 10 to 70 wt %, 10 to 60 wt %, 10 to 50 wt %, 10 to 30 wt %, 25 to 99 wt %, 25 to 95 wt %, 25 to 90 wt %, 25 to 80 wt %, 25 to 70 wt %, 25 to 60 wt %, 25 to 50 wt %, 25 to 30 wt %, 40 to 99 wt %, 40 to 95 wt %, 40 to 90 wt %, 40 to 80 wt %, 40 to 70 wt %, 40 to 60 wt %, 40 to 50 wt %, 50 to 99 wt %, 50 to 95 wt %, 50 to 90 wt %, 50 to 80 wt %, 60 to 99 wt %, 60 to 95 wt %, 60 to 90 wt %, or 60 to 80 wt %, but is not limited thereto.

There may be no particular limitation on the type of the food composition. Examples of the food to which the food composition may be added may include dairy products including ice creams, various soups, beverages, teas, drinks, alcoholic beverages, vitamin complexes, and the like, and may be prepared as powders, granules, tablets, capsules, syrups, or beverages, may be prepared in a known form without limitation, and may include all foods designed to sufficiently express a body control function related to biodefense rhythm control, disease prevention and recovery, and the like, of a food group or food composition in which an added value is given to act and express the function of the corresponding food for a specific purpose in a conventional sense.

The food composition may further contain at least one additive selected from the group consisting of various nutrients, vitamins, minerals (electrolytes), flavoring agents such as synthetic flavoring agents, natural flavoring agents or the like, coloring agents, pectic acid or a salt thereof, alginic acid or a salt thereof, organic acids, protective colloidal thickeners, pH adjusters, stabilizers, preservatives, glycerin, alcohols, carbonating agents used in carbonated beverages, natural fruit juices, fruit flesh for the preparation of fruit beverages and vegetable beverages, etc. The ratio of these additives may be generally selected in the range of about 0.0001 to about 20 parts by weight per 100 parts by weight of the total health functional food, but is not limited thereto.

As used herein, the term “prevention” may be meant to prevent a decrease in the ocular capacity beforehand by applying the Achyranthes japonica, the Glycyrrhiza uralensis, or the mixed extract thereof according to the present invention to a food composition, and the term “amelioration” may be meant to promote, enhance, or strength the ocular capacity of the composition of the present invention.

Another embodiment of the present invention is to provide a pharmaceutical composition for preventing or treating eye diseases, including Achyranthes japonica, Glycyrrhiza uralensis, or a mixed extract thereof as an active ingredient.

In addition, the present invention may provide a method for preventing or treating eye diseases, including administering a pharmaceutical composition including Achyranthes japonica, Glycyrrhiza uralensis, or a mixed extract thereof into an individual.

* In the present specification, the terms “Achyranthes japonica,” “Glycyrrhiza uralensis,” “mixed extract,” “eye disease,” and “prevention” may be the same as described above.

As used herein, the term “treatment” may be meant to include all of the followings: alleviation or amelioration of symptoms, reduction in the scope of disease, delay or alleviation of disease progression, amelioration, alleviation or stabilization of a disease state or symptoms, partial or complete recovery, prolongation of survival, other beneficial treatment results, and the like. “Prevention” may be meant to include all mechanisms and/or effects which act on a subject who does not have a specific symptom or disease to prevent the occurrence of the specific symptom or disease or delay an occurrence time thereof.

As used herein, “administration” may be meant to provide a pharmaceutically effective amount of the pharmaceutical composition provided herein to an individual by any suitable method.

The pharmaceutical composition may be administered to mammals including humans, dogs, cats, horses, cows, pigs, goats, rabbits, mice, rats, etc., or cells, tissues, or cultures thereof isolated therefrom via various routes. The administration method may include any method commonly used, for example, oral administration, or parenteral administration such as intravenous administration, muscular administration, subcutaneous administration, intraperitoneal administration, rectal administration, and local administration to a lesion site. The pharmaceutical composition may be used by being formulated into oral formulations such as powders, granules, tablets, capsules, suspensions, emulsions, syrups, aerosols, etc., parenteral formulations in the form of transdermal agents, suppositories, sterile injectable solutions, etc., according to conventional methods.

The pharmaceutical composition may further contain an adjuvant such as a pharmaceutically and/or physiologically acceptable carrier, excipient, diluent and/or the like in addition to the mixed extract of Achyranthes japonica and Glycyrrhiza uralensis as an active ingredient. Examples of the carrier, excipient, or diluent may include at least one selected from the group consisting of lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, tale, magnesium stearate, mineral oil, and the like. In the case of formulating, at least one diluent or excipient selected from the group consisting of commonly used fillers, extenders, binders, wetting agents, disintegrants, surfactants, etc. may be used. Solid preparations for oral administration may include at least one selected from the group consisting of tablets, pills, powders, granules, capsules, syrups, powders, suspensions, etc., and such solid preparations may be prepared by mixing the extract with at least one excipient, for example, at least one selected from the group consisting of starch, calcium carbonate, sucrose, lactose, gelatin, etc. In addition, lubricants such as magnesium stearate and talc may be also used in addition to simple excipients. A liquid preparation for oral administration may correspond to at least one selected from the group consisting of suspensions, solutions for internal use, emulsions, syrups, and the like, and may include at least one selected from the group consisting of various excipients, for example, wetting agents, sweeteners, fragrances, preservatives, and the like in addition to water and liquid paraffin, which are commonly used simple diluents. A preparation for parenteral administration may include at least one selected from sterile aqueous solutions, non-aqueous solvents, suspensions, emulsions, lyophilized preparations, suppositories, transdermal agents, etc. The non-aqueous solvent and the suspending agent used herein may include at least one selected from the group consisting of propylene glycol, polyethylene glycol, vegetable oil like olive oil, injectable ester like ethyl oleate, etc.

The pharmaceutical composition may be administered in a pharmaceutically effective amount. The term “pharmaceutically effective amount” may refer to an administration dose of an active ingredient capable of exerting a beneficial effect (e.g., prevention, alleviation, amelioration, reduction, suppression, removal, and/or treatment) on symptoms and/or diseases, may be prescribed in various ways by various factors such as a formulation method, an administration method, a patient's age, weight, gender, pathological condition, food, administration time, administration interval, administration route, excretion rate, and response sensitivity, and may be administered once or in divided doses a day at regular time intervals according to the judgment of a doctor or a pharmacist.

The pharmaceutical composition may be administered as an individual therapeutic agent or in combination with other therapeutic agents, and may be administered sequentially or simultaneously with a conventional therapeutic agent. In addition, the pharmaceutical composition may be administered in a single or multiple manner. Considering all the above factors, the pharmaceutical composition may be administered in such an amount that a maximum effect may be achieved by a minimum amount without a side effect, which may be easily determined by those skilled in the art. In addition, the pharmaceutically effective amount may vary depending on the individual to be administered.

In one specific embodiment, the dosage (pharmaceutically effective amount) of the pharmaceutical composition may be

1 mg/kg/day or more, 2 mg/kg/day or more, 3 mg/kg/day or more, 4 mg/kg/day or more, 5 mg/kg/day or more, 6 mg/kg/day or more, 7 mg/kg/day or more, 8 mg/kg/day or more, 9 mg/kg/day or more, or 10 mg/kg/day or more; and/or 15 mg/kg/day or less, 30 mg/kg/day or less, 50 mg/kg/day or less, 90 mg/kg/day or less, 120 mg/kg/day or less, or 150 mg/kg/day or less, based on the weight of the mixed extract of Achyranthes japonica and Glycyrrhiza uralensis as an active ingredient.

In one embodiment, the pharmaceutical composition may be administered at a dose of 50 to 150 mg/kg/day, 60 to 140 mg/kg/day, 70 to 130 mg/kg/day, 80 to 120 mg/kg/day, 90 to 110 mg/kg/day, or about 100 mg/kg/day, based on the weight of the mixed extract of Achyranthes japonica and Glycyrrhiza uralensis, but is not limited thereto.

The composition including the Achyranthes japonica, the Glycyrrhiza uralensis, or the mixed extract thereof as an active ingredient may be administered to a subject by various routes. All modes of administration may be expected, and may include, for example, oral, rectal or intravenous, intramuscular, or subcutaneous administration.

In the present specification, the term “individual” may refer to all the animals such as rats, mice, livestock, etc., including humans, who have already developed or are likely to develop eye diseases, and specifically may refer to mammals including humans, but is not limited thereto.

In addition, the pharmaceutical composition may be used alone or in combination with methods of using a diet, physical therapy, and/or a biological response modifier for the prevention, amelioration, and/or treatment of eye diseases.

Still another embodiment of the present invention may provide a quasi-drug composition for preventing or ameliorating eye diseases, including Achyranthes japonica, Glycyrrhiza uralensis, or a mixed extract thereof as an active ingredient.

In the present specification, the terms “Achyranthes japonica,” “Glycyrrhiza uralensis,” “mixed extract,” “eye disease,” “prevention” and “amelioration” may be the same as described above.

In the present invention, the term “quasi-drug composition” may include textile and rubber products for diagnosing, treating, and ameliorating diseases of humans or animals, articles other than appliances or machines, fungicides/insecticides, and the like for preventing infectious diseases, which have minor or no direct action on the human body. The type or formulation of the quasi-drug composition of the present invention may not be particularly limited, but specifically may be an eye cleansing agent or an eye ophthalmic solution.

Still another embodiment of the present invention may provide a feed composition for preventing or ameliorating eye diseases, including Achyranthes japonica, Glycyrrhiza uralensis, or a mixed extract thereof as an active ingredient.

In the present specification, the terms “Achyranthes japonica,” “Glycyrrhiza uralensis,” “mixed extract,” “eye disease,” “prevention” and “amelioration” may be the same as described above.

As used herein, the term “feed” may refer to any natural or artificial diet, single meal, etc., or an ingredient of the single meal for animals for or suitable for eating, taking in, and digesting.

The kind of the feed is not particularly limited, and may include any feed commonly used in the art. Non-limiting examples of the feed may include plant feeds such as grains, root fruits, food processing by-products, algae, fibers, pharmaceutical by-products, oils and fats, starches, gourds, grain by-products or the like; and animal feeds such as proteins, minerals, oils and fats, minerals, unicellular proteins, animal planktons, food products, or the like. These may be used alone or in combination of two or more.

The extract of Achyranthes japonica and Glycyrrhiza uralensis or a fraction thereof according to the present invention included in the feed composition of the present invention may vary depending on the purpose and conditions of using the feed, and may be included, for example, in an amount of 0.01 to 100 wt %, more specifically 1 to 80 wt % based on the total weight of the livestock feed composition, but is not limited thereto.

### MODE FOR INVENTION

Hereinafter, the present invention will be described in more detail through exemplary embodiments. These Examples are intended to explain the present invention more specifically, but the following Examples, Comparative Examples, Experimental Examples, and Preparation Examples are only illustrative of the contents of the present invention, and the scope of the present invention is not limited to the following Examples, Comparative Examples, Experimental Examples, and Preparation Examples. Examples, Comparative Examples, Experimental Examples, and Preparation Examples of the present invention are provided to more completely explain the present invention to those of ordinary skill in the art.

### Experimental Example 1. Preparation of Achyranthes Japonica, Glycrrhiza Uralensis, or Mixed Extract Thereof

Achyranthes japonica and Glycyrrhiza uralensis were mixed in the ratios shown in Table 1 below and subjected to reflux extraction with 30% water-soluble ethanol and 50% water-soluble ethanol at 50° C. for two hours. At this time, the volume of water-soluble ethanol was about 10 times the amount of herbal medicine. The herbal medicine was about 300 g and subjected to extraction with about 300 ml of water-soluble ethanol. After the reflux extraction twice, the first and second extracts were combined and concentrated under reduced pressure at 45 to 50° C. and then dried in a freeze dryer (Table 1).

TABLE 1

| No. | Experimental Conditions |
|---|---|
| JK-502 | 30% 1:6 (Achyranthes japonica:Glycyrrhiza uralensis = 1:6) |
| JK-503 | 30% 6:1 (Achyranthes japonica:Glycyrrhiza uralensis = 6:1) |
| JK-504 | 30% 1.9 (Achyranthes japonica:Glycyrrhiza uralensis = 1:9) |
| JK-505 | 50% 1:6 (Achyranthes japonica:Glycyrrhiza uralensis = 1:6) |
| JK-506 | 50% 6:1 (Achyranthes japonica:Glycyrrhiza uralensis = 6:1) |
| JK-507 | 50% 1:1 (Achyranthes japonica:Glycyrrhiza uralensis = 1:1) |
| JK-508 | 50% 1:3 (Achyranthes japonica:Glycyrrhiza uralensis = 1:3) |
| JK-509 | 50% 1:9 (Achyranthes japonica:Glycyrrhiza uralensis = 1:9) |
| JK-510 | 50% 3:1 (Achyranthes japonica:Glycyrrhiza uralensis = 3:1) |
| JK-511 | 30% 1:1 (Achyranthes japonica:Glycyrrhiza uralensis = 1:1) |
| JK-512 | 30% 1:3 (Achyranthes japonica:Glycyrrhiza uralensis = 1:3) |
| JK-514 | 50% Glycyrchiza uralensis |
| JK-516 | 50% 9:1 (Achyranthes japonica:Glycyrrhiza uralensis = 9:1) |
| JK-516 | 30% 3:1 (Achyranthes japonica:Glycyrrhiza uralensis = 3:1) |
| JK-517 | 30% 9:1 (Achyranthes japonica:Glycyrrhiza uralensis = 9:1) |
| JK-519 | 30% Achyranthes japonica |
| JK-520 | 50% Achyranthes japonica |

### Experimental Example 2. Cell Cultivation

Human retinal pigmented epithelial cell lines (ARPE-19 cells) were purchased from American Type Culture Collection (Manassas, VA, USA), and 10% fetal bovine serum (Gibco, Burlington, ON, Canada) and 1% penicillin-streptomycin (Gibco, Life Technologies Limited, Paisley, UK) were added to DMEM/F12 (Gibco, Carlsdad, CA, USA), which is a mixed medium of Dulbeccos modified Eagle® s medium (DMEM) and Ham's F12 at 1:1, and subjected to wet cultivation under the conditions of 37° C. and 5% CO₂. After that, when grown to a density of 70% or more, they were subcultured and used in the experiment.

### Experimental Example 3. Confirmation of Cell Viability in Human Retinal Epithelial Cell Line (ARPE-19)

In order to evaluate the cytotoxicity of the candidate materials, cell counting kit-8 (CCK-8; Dojindo, Japan) assay was performed. First, in order to confirm the cytotoxicity of the candidate materials per se, when ARPE-19 cells were dispensed into 96 well plate (1×10⁴ cells/well) and reached 70% of confluence, the candidate materials for each concentration were treated and cultured for 24 hours. Subsequently, 20 μl of CCK-8 solution was added to each well, and then cultured in an incubator at 37° C. and 5% CO₂ for two hours. Then, an absorbance value was measured at 450 nm using a microplate reader (Molecular Devices, Sunnyvale, CA, USA). Meanwhile, in order to confirm a cell protective effect of the candidate materials against oxidative stress according to the treatment with tert-butyl hydroperoxide (tBHP, Sigma-Aldrich Chemical Co., USA), tBHP (300 μM) and 17 extracts (200 μg/ml) were simultaneously treated for 24 hours, and each well was treated with 20 μl of CCK-8 to measure an absorbance value at 450 nm using a microplate reader.

### Experimental Example 4. Confirmation of Antioxidant Effect in Retinal Epithelial Cell Line

In order to confirm if the Achyranthes japonica, the Glycyrrhiza uralensis, or the mixed extract thereof protects ARPE-19 cells from oxidative stress caused by tert-butyl hydroperoxide (tBHP), 300 μM tBHP and 17 kinds of extracts (200 vg/ml) were simultaneously treated to confirm the cell viability in 24 hours later. The demonstrated efficacy at concentration 200 μg/ml reconfirmed efficacy at lower concentrations of 1, 10, 100, 200 μg/ml.

### Experimental Example 5. Measurement of Oxygen Consumption Rate (OCR) and ATP Production of Mitochondria

In order to measure an intracellular oxygen consumption rate, ARPE-19 cells were cultured at 1×10⁴ cells/well in a plate for XF analyzer (Seahorse Bioscience, USA) using DMEM/F-12 medium containing 10% fetal bovine serum (GIBCO, USA). In order to measure an oxygen consumption rate (OCR) of the cells, the cells were cultured in a CO₂-free cell incubator for one hour after being replaced with bicarbonate-free medium. After measuring an OCR value at a basal level of the cells for 20 minutes, the cells were simultaneously treated with 300 μM tBHP or each candidate material for four hours, after which an OCR change was measured for 60 minutes. After that, an amount of the OCR change was measured again while being sequentially treated with four respiratory chain inhibitors such as 0.5 μM oligomycin, 1 μM carbonyl cyanide-4-(trifluoromethoxy)phenylhydrazone (FCCP), 1 μM rotenone, 1 μg/ml antimycin A, etc.

### Experimental Example 6. Measurement of Intracellular Mitochondrial Morphology and Reactive Oxygen

In order to measure a mitochondrial morphology and an intracellular reactive oxygen species (ROS) due to intracellular oxidative stress, ARPE-19 cells were cultured at 1.5×10⁴ cells/well in μ-slide well plate using DMEM/F-12 medium containing 10% fetal bovine serum (GIBCO, USA). After that, the cells were simultaneously treated with 300 μM tBHP or each candidate material for four hours, after which a 2′,7′-dichlorodihydrofluorescein diacetate (DCF-DA; Invitrogen) staining method was used to measure a change in ROS production, and MitoTracker™ Red CMX_Ros (Thermo Fisher Scientific Inc., USA) staining was performed for 40 minutes to confirm the mitochondrial morphology. Then, washing was performed twice with PBS and replaced with a medium containing DAPI. After 30 minutes, washing was performed twice with PBS and replaced with a medium, and images were obtained using a confocal laser scanning microscope (LSM 710; Carl Zeiss, Inc.). ROS intensity was measured by an ROS detection cell-based assay kit (Cayman Chemical Inc., USA), and mitochondria intensity was measured with a fluorescence microplate reader in the range of kex 556 and λem 575 nm after being subjected to cell lysis.

### Experimental Example 7. Statistical Processing

Statistical analysis was performed using one-way ANOVA of GrapbPad Prism version 7.04 (Graphpad Inc., San Diego, CA, USA), and p<0.05 values were reported as significant values by post-hoc testing with T-test, and subjected to statistical processing.

### Example 1. Reflux Extract in Aqueous Ethanol Solution

The results of extraction, concentration, and drying according to above Experimental Example 1 were derived and summarized in Table 2 as follows (Table 2).

### Example 2. Confirmation of Cytotoxicity in Human Retinal Epithelial Cell Line (ARPE-19)

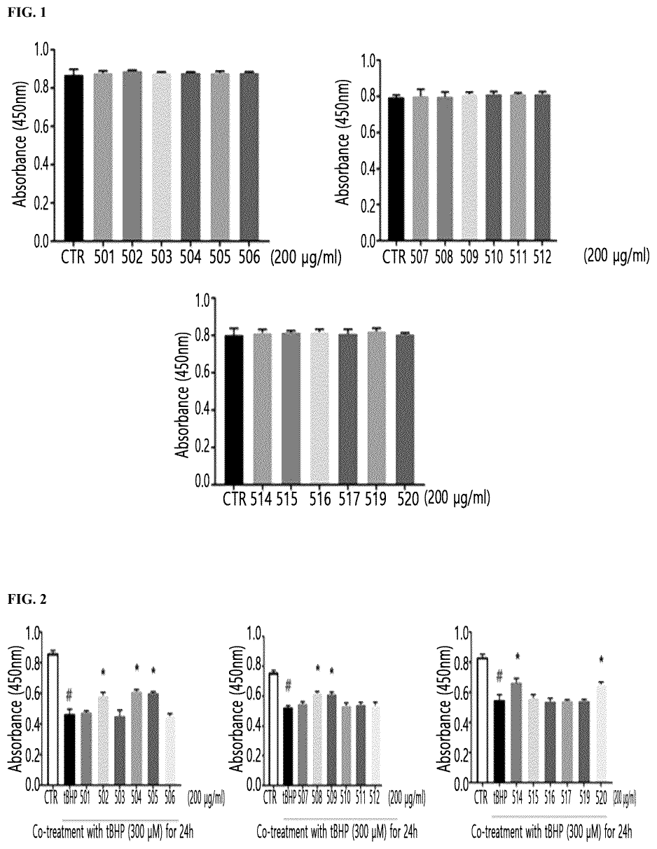

As a result of confirming cell viability in the human retinal epithelial cell line (ARPE-19), it was confirmed that the Achyranthes japonica, the Glycyrrhiza uralensis, or the mixed extract thereof (200 μg/ml) is not cytotoxic in the ARPE-19 cell line (FIG. 1), as can be seen in FIG. 1.

### Example 3. Confirmation of Antioxidant Effect in Retinal Epithelial Cell Line

As a result of confirming an antioxidant effect of the Achyranthes japonica, the Glycyrrhiza uralensis, or the mixed extract thereof according to the present invention in retinal epithelial cell lines, it was confirmed (FIG. 2) that the mixed extracts JK-502, 504, 508, and 509, the Glycyrrhiza uralensis extract 514, and the Achyranthes japonica extract 520 (200 μg/ml) have antioxidant efficacy of protecting ARPE-19 cells from oxidative stress caused by tBHP (300 uM), as can be seen in FIG. 2.

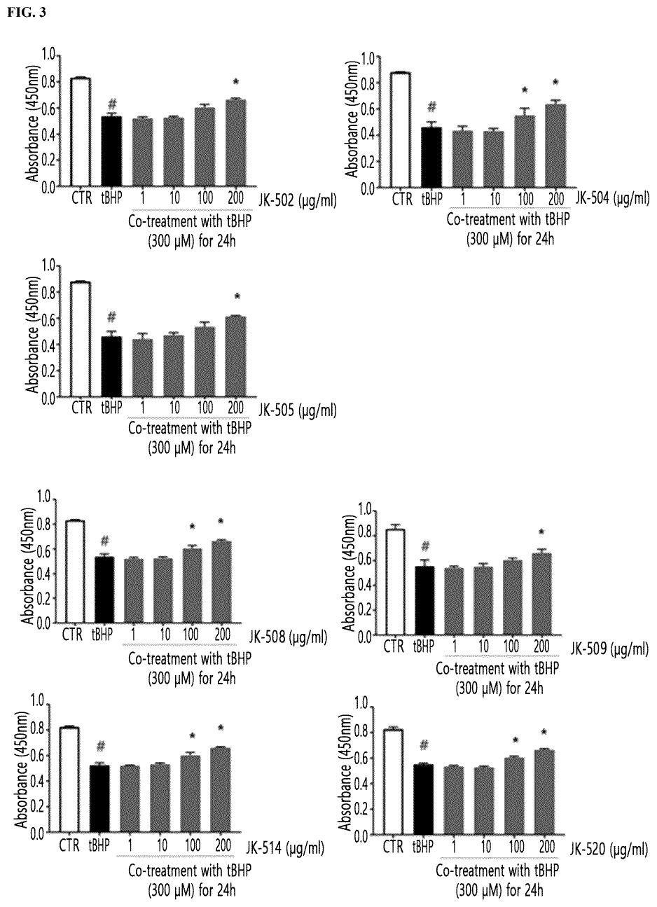

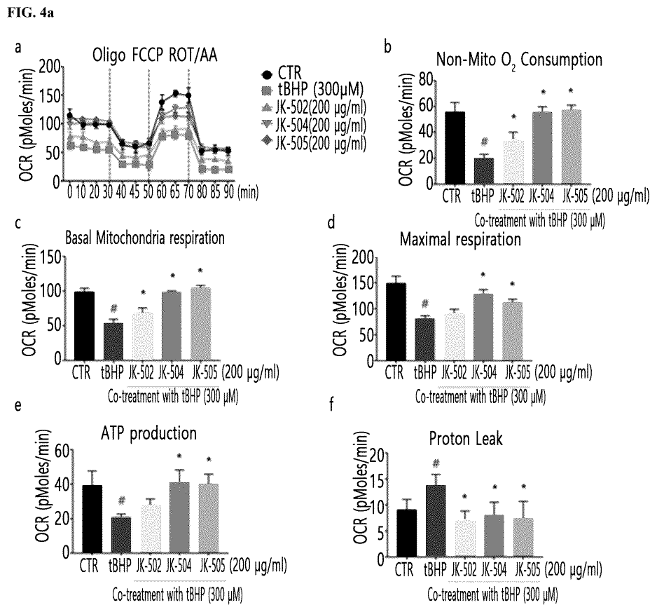

As a result of confirming the efficacy of the extracts with antioxidant efficacy verified at 200 μg/ml at a lower concentration (1, 10, 100, 200 vg/mW), it was confirmed that antioxidant effects are exhibited at 100, 200 μg/ml of the extracts 504, 508, 514, and 520 and at 200 μg/ml of the extracts 502, 505, and 509, respectively (FIG. 3).

In particular, in each of the Achyranthes japonica extract and the Glycyrrhiza uralensis extract, it was confirmed that the antioxidant effect is very excellent only when being extracted with 50% aqueous ethanol solution, though there is no antioxidant effect in retinal epithelial cell lines when being extracted with 30% aqueous ethanol solution. Even in the mixed extract of Achyranthes japonica and Glycyrrhiza uralensis, it was confirmed that there is no effect when Achyranthes japonica and Glycyrrhiza uralensis are mixed in an equivalent ratio, and there is no antioxidant effect even when Achyranthes japonica is mixed in a large weight ratio compared to Glycyrrhiza uralensis. On the contrary, it was confirmed that the antioxidant effect is achieved in the retinal epithelial cell lines when Glycyrrhiza uralensis is mixed in a large weight ratio compared to Achyranthes japonica. Among them, it was confirmed that a very excellent antioxidant effect is achieved in a mixing weight ratio of 1:3 to 1:9.

Accordingly, it can be confirmed that the effects vary depending on the content of ethanol in the aqueous solution, out of which individual extracts have excellent effects in 50% aqueous ethanol solution, and it can be confirmed that the mixed extract has an effect when extracted with 30 to 50% aqueous ethanol solution, but has an effect in a specific range of the mixing ratio of Achyranthes japonica and Glycyrrhiza uralensis.

### Example 4. Effect of Restoring Oxygen Consumption and ATP Production in Mitochondria

The effects of the JK-502, 504, 505, 508, 509, 514, and 520 extracts, which were confirmed to have antioxidant effects in Example 3, were also confirmed on mitochondrial stress responses caused by oxidative stress.

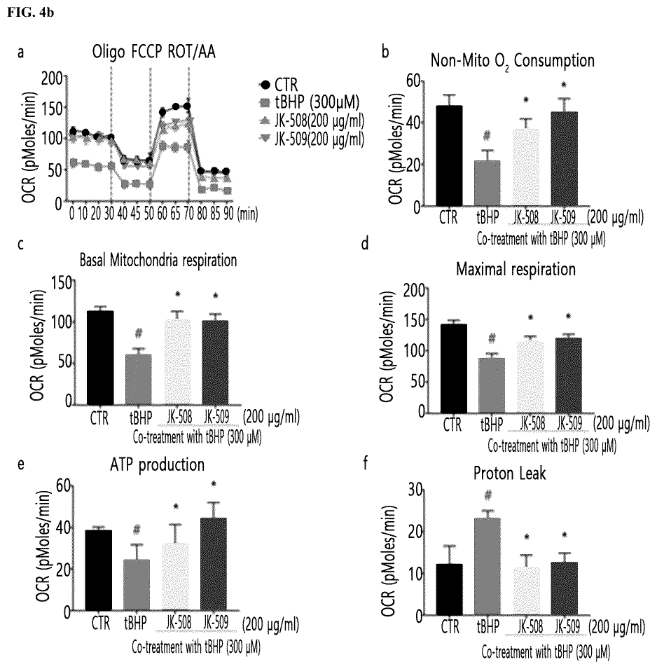

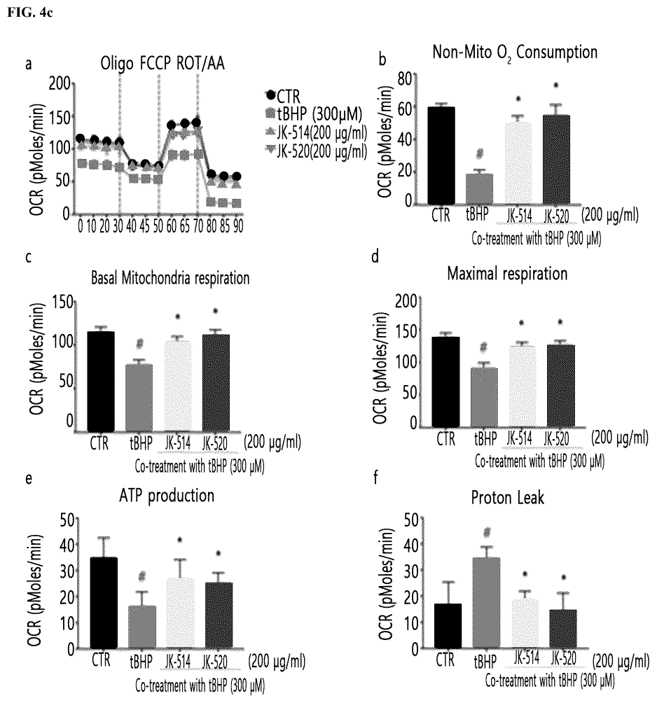

As a result, as can be seen in FIG. 4, in the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention, it was confirmed that a reduced oxygen consumption rate (OCR) in mitochondria is significantly restored and at the same time, the reduced ATP productivity is restored to almost normal levels in JK-504, 505, 508, 509, 514, and 520. In addition, it was confirmed that a proton leak increased by oxidative stress is significantly reduced by each extract (FIG. 4).

This suggests that the Achyranthes japonica, the Glycyrrhiza uralensis or the mixed extract thereof according to the present invention has effects of restoring oxygen consumption and ATP production in mitochondria, thereby providing effects of preventing, ameliorating, and treating eye diseases.

### Example 5. Mitochondria Protective Effect

Previously, mitotracker and DCFDA reagents were used to qualitatively and quantitatively confirm the effect of each extract on mitochondrial damage caused by oxidative stress in the extract exhibiting a cell protective effect by oxidative stress.

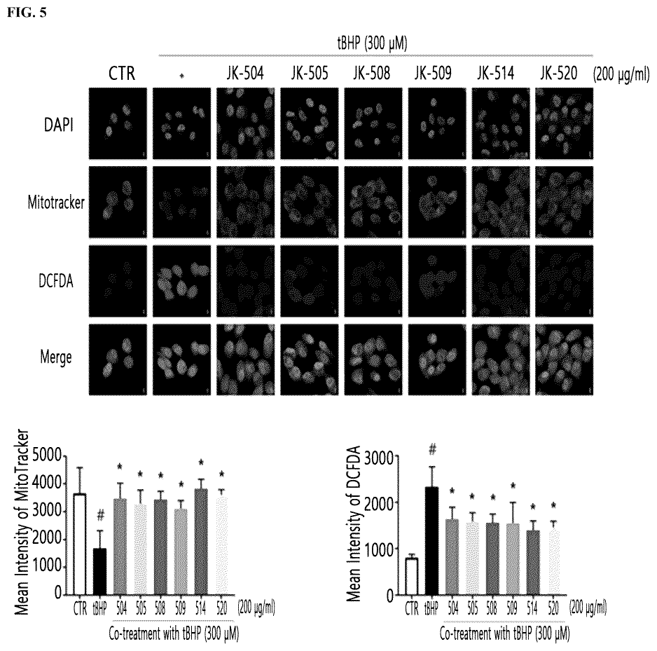

Specifically, as a result of confirming the intensity of mitochondria by mitotracker, as can be seen in FIG. 5, it was qualitatively and quantitatively confirmed that the intensity of mitotracker reduced by tBHP is restored in the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention.

At the same time, as a result of measuring an intracellular active oxygen by a fluorescence analysis method in which DCFDA is oxidized to DCF by the active oxygen and takes on a green color, it was confirmed that the intensity is remarkably reduced in a group treated with the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention (FIG. 5), whereas the intensity of DCFDA is strongly exhibited due to the oxidative stress increased in the cell by tBHP.

This also suggests that the Achyranthes japonica, the Glycyrrhiza uralensis or the mixed extract thereof according to the present invention has effects of restoring oxygen consumption and ATP production in mitochondria, thereby providing effects of preventing, ameliorating, and treating eye diseases.

### Example 6. Effect of Inhibiting Retinal Cell Death by Blue Light Oxidative Stress

Using zebrafish as an animal model, it was confirmed that the Achyranthes japonica, the Glycyrrhiza uralensis or the mixed extract thereof according to the present invention has the effect of inhibiting retinal cell death on damage to retinal cells by blue light oxidative stress.

The oxidative stress caused by blue light does a damage to retinal pigment epithelium (RPE) cells and photoreceptor cells, and a photoreceptor layer is an outer nuclear layer and includes rod cell nuclei and cone cell nuclei.

Specific experimental methods are as follows.

In the case of zebrafish, its zebrafish system was maintained using Westerfield's husbandry technique, subjected to light control under a light condition for 14 hours and under a dark condition for 10 hours (14/10 light/dark cycle) under a temperature of 28.5° C. and 200 Lux lighting, and fed with a certain amount of mixed commercial diet of fish meal, shrimp meal, and krill meal once or twice a day.

Then, the feed from the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention were used by being dissolved in a PBS solvent, and N-acetylcysteine was selected as a positive control group and confirmed together.

30 Zebrafish, which were six-month-old adults after spawning, were transferred to a 3 L water bath with an underwater warmer (28.5° C.) installed therein, and transferred to a darkroom for dark adaptation for 48 hours. Feed was supplied once during the dark adaptation.

Three dark-adapted zebrafish were divided into a light-treated group, an N-acetylcysteine group, and JK502, JK 504, JK505, JK508, JK509, JK514, and JK520 groups of the present invention, respectively, and were divided into 200 mL system water using a 500 mL beaker, and blue light stress was applied using a blue light emitting diode lamp (Blue Light, 440 nm, 12,000 Lux) for 24 hours. The system water was equally adjusted to a volume of 200 mL, considering the amount of solvent used for each group, and at this time the extract of the present invention and each concentration were equally used for three zebrafish at a concentration of 100 ag with regard to system tank water.

After the end of exposure to a light source, the zebrafish were euthanized using ice-cold water mixed with ice, fixed in a 70% aqueous ethanol solution for 24 hours, and then put again in 4% parafomaldehyde solution and fixed at room temperature for 24 hours or more. After that, in order to prepare a tissue sample for an ocular tissue examination of zebrafish, an eyeball was extracted with a fine tweezers and made into a paraffin block, and a retinal tissue including an optic disc and close to the optic nerve for each group was selected to prepare a 4 μm tissue section, which was then subjected to H&E staining.

Then, a change in the tissue structure of the H&E-stained retina was observed for each test group through a light source microscope (KB-600 Biological Microscope, KCS3-63SS Sony CMOS color camera).

At this time, a tissue including optic nerve was selected from among H&E-stained ocular tissue sections, so as to measure a thickness of an outer nuclear layer, an inner nuclear layer, an inner plexiform layer, and whole retinal (NFL-RPE) at a distance of 100 μm from the optic nerve.

An Excel function was used for statistical analysis to confirm that the ONL thickness and the number of ONI cell nuclei for each group of zebrafish included in the experiment were statistically significant. A one-way analysis of variance was performed to compare the results between the groups, and it was judged statistically significant that a p-value is less than 0.05 in the analysis results.

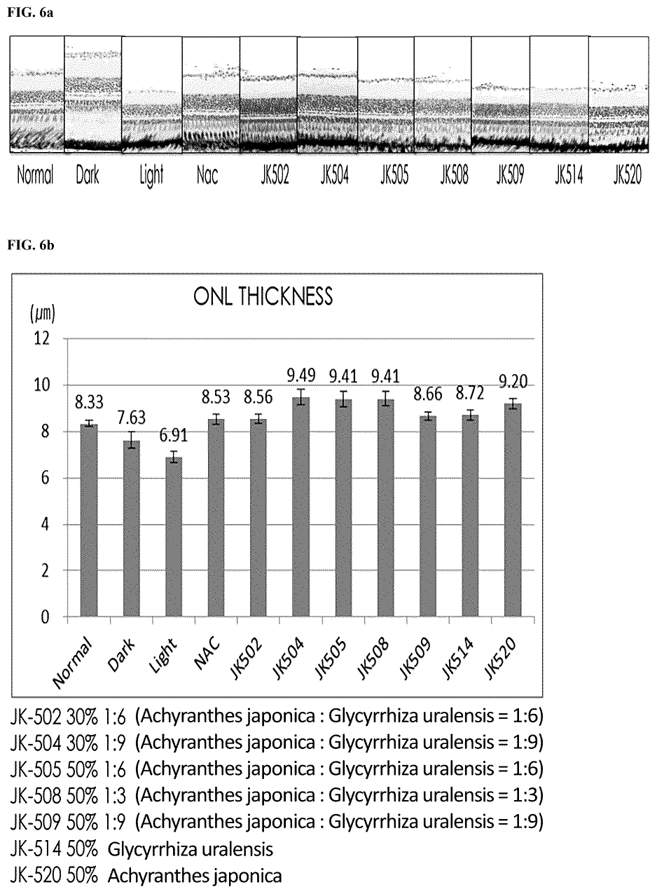

The experimental results are shown in FIGS. 6 to 9.

As a result, compared to the retinal tissue of the zebrafish after dark adaptation, it was confirmed that an overall thickness of the outer nuclear layer, the inner nuclear layer, the inner plexiform layer and the whole retinal of the zebrafish under the stress of the blue light is significantly reduced all in the normal state and the dark adaptation state.

On the contrary, as can be seen in FIG. 6, it was confirmed that the thickness of the outer nuclear layer (ONL) is remarkably increased from 6.91±0.23 μm, which is the thickness of the ONL under blue light stress, by 8.56±021 μm (19.3%), 9.49±0.33 μm (27.2%), 9.41+033 μm (26.6%), 9.41±030 μm (26.6%), and 8.66±0.18 μm, respectively, in JK 502, JK 504, JK 505, JK 508, and JK 509, which are the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention. In the case of single extracts, it was also confirmed that the thickness is also increased to 8.72 μm and 9.20 μm in the Glycyrrhiza uralensis extract and the Achyranthes japonica extract, respectively, thus suggesting that these effects are similar to or superior to NAC which is a positive control (FIG. 6).

In particular, in the mixed extracts of JK 504, JK 505, and JK 508 in which Achyranthes japonica and Glycyrrhiza uralensis were mixed at a mixing ratio of 1:3 to 1:9, it was confirmed that the thickness of the outer nuclear layer is excellently increased compared to the thickness of the single extracts of JK 514 and JK 520, thus suggesting that the effect of inhibiting retinal cell death is greater in the mixed extract compared to each single extract.

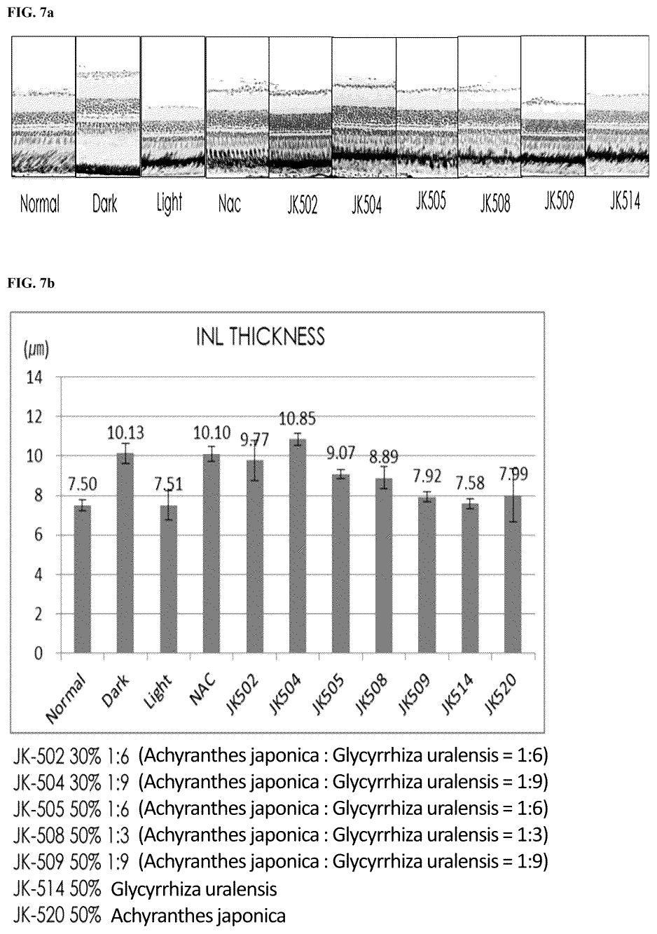

Then, as can be seen in FIG. 7, it was confirmed that the thickness of the inner nuclear layer (INL) under blue light stress is 7.51±0.73 μm and the thickness is increased to 9.77±1.04 μm, 10.85±0.32 μm, 9.07±0.21 μm, 8.89±0.56 μm, and 7.92±0.27 μm, respectively, in the groups treated with JK 502, JK 504, JK 505, JK 508, and JK 509, which are the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention.

It can also be confirmed that the effect is more excellent in the mixed extract in which Achyranthes japonica and Glycyrrhiza uralensis are mixed at a ratio of 1:3 to 1:9 compared to the single extract of Achyranthes japonica and Glycyrrhiza uralensis.

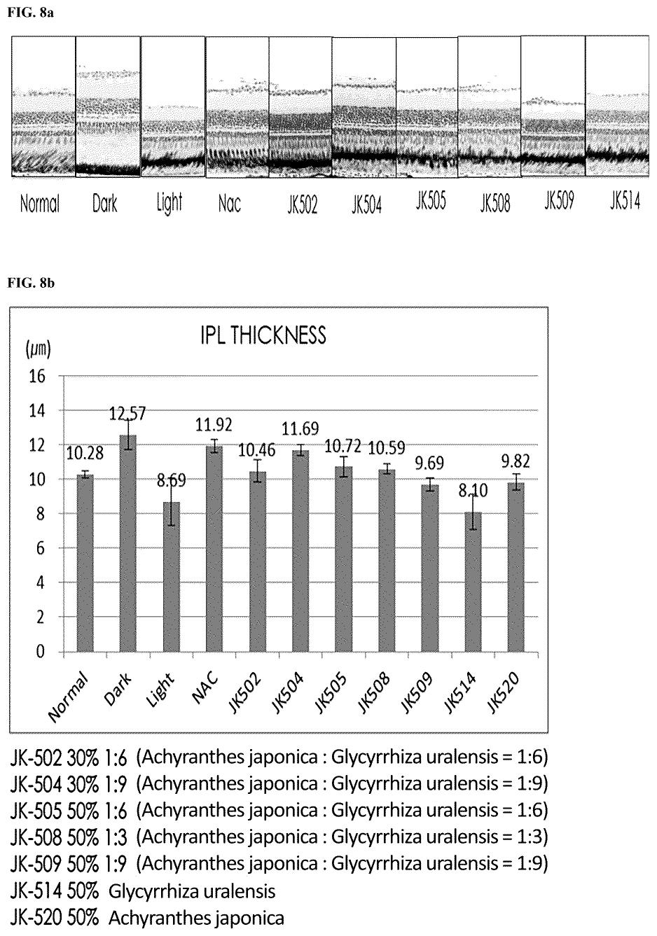

Then, as can be seen in FIG. 8, it was confirmed that the thickness of the inner plexiform layer (IPL) under blue light stress is 8.69±1.40 μm and the thickness is increased to 10.46±0.64 μm (16.9%), 11.69±0.33 μm (25.75%), 10.72±0.61 am (18.9%), 10.59=0.29 qm (17.9%), and 9.69±0.40 tam (10.3%), respectively, in JK 502, JK 504, JK 505, JK 508, and JK 509, which are the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention, thus confirming that this also has an effect similar to or better than the positive control.

In particular, in the case of the thickness of the inner plexiform layer (IPL), it can also be confirmed that the effect is more excellent in the mixed extract in which Achyranthes japonica and Glycyrrhiza uralensis are mixed at a ratio of 1:3 to 1:9 compared to the single extract of Achyranthes japonica and Glycyrrhiza uralensis.

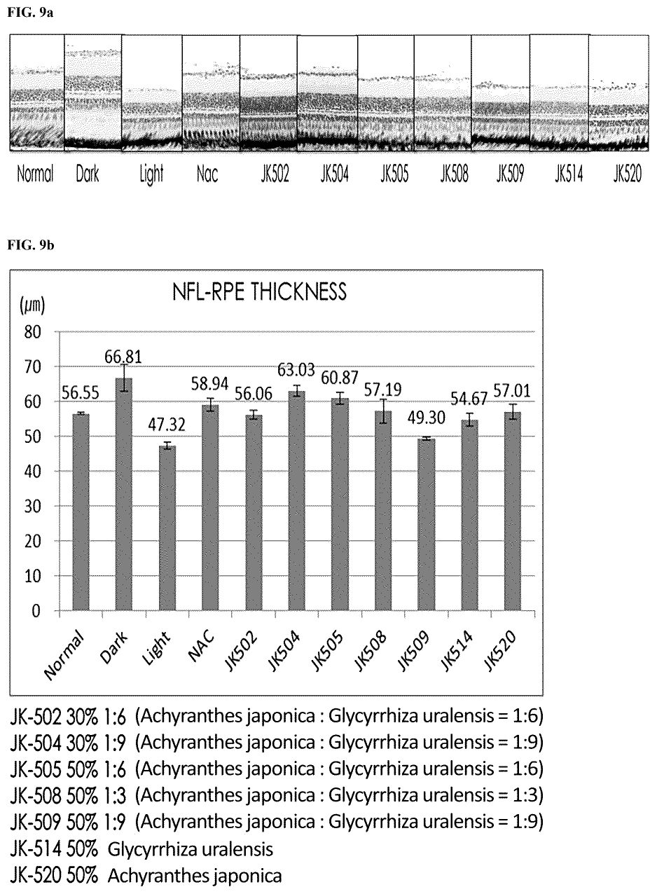

Finally, as can be seen in FIG. 9, it can be confirmed that an overall thickness of the retina from the neural layer of retina (NFL) to the retinal pigmented epithelial cell layer (RPE) is measured to be 47.32±1.10 μm under blue light stress after dark adaptation, but it is remarkably increased compared to the values measured under blue light stress in all of JK 502, JK 504, JK 505, JK 508, JK 509, JK514, and JK520, which are the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention, out of which it is increased to 63.03±1.58 μm in a group treated with JK 504, which is a mixed extract, thus providing a very excellent increasing effect of 25.5%, which is excellent compared to the positive control.

Overall, it was confirmed that the Achyranthes japonica extract and the Glycyrrhiza uralensis extract obtained with 50% aqueous ethanol solution, and the mixed extract of Achyranthes japonica and Glycyrrhiza uralensis at 1:3 to 1:9 obtained with 30 to 50% aqueous ethanol solution according to the present invention, which are the active ingredients through Examples 1 to 6, have excellent antioxidant effects in retinal epithelial cells and excellent effects of restoring oxygen consumption and ATP production in mitochondria, and furthermore, have an effect of protecting mitochondria and effectively suppressing retinal cell death due to blue light oxidative stress, thus suggesting that these extracts may be utilized as drugs, health functional foods, and the like for preventing, ameliorating, or treating eye diseases. In addition, it can be confirmed that there are advantages in that it needs a less number of medicinal ingredients essentially included therein and has a less drug interaction, thus providing excellent commercial viability and a row risk of side effects.

A formulation example of the composition according to one embodiment of the present invention will be described below, but may be applied to other various formulations, and this is not intended to limit the present invention, but to specifically describe the present invention.

### Preparation Example 1. Preparation of Pharmaceutical Preparation

### 1-1. Preparation of Powder

First, 100 mg of the mixed extract of Achyranthes japonica and Glycyrrhiza uralensis prepared in any one of Examples 1.3 to 1.8, 100 mg of lactose, and 10 mg of talc were mixed and then filled in an airtight package to prepare a powder.

### 1-2. Preparation of Tablet

A tablet was prepared by mixing 100 mg of the mixed extract of Achyranthes japonica and Glycyrrhiza uralensis prepared in any one of Examples 1.3 to 1.8, 100 mg of corn starch, 100 mg of lactose, and 2 mg of magnesium stearate, and then tableting the resulting mixture according to a conventional tablet preparation method.

### 1-3. Preparation of Capsule

A capsule was prepared by mixing 100 mg of the mixed extract of Achyranthes japonica and Glycyrrhiza uralensis prepared in any one of Examples 1.3 to 1.8, 100 mg of corn starch, 100 mg of lactose, and 2 mg of magnesium stearate, and filling the resulting mixture in a gelatin capsule.

### 1-4. Preparation of Injection

First, 100 mg of the mixed extract of Achyranthes japonica and Glycyrrhiza uralensis prepared in any one of Examples 1.3 to 1.8, an appropriate amount of sterile distilled water for injection, and an appropriate amount of a pH adjuster were mixed per ampule (2 mL) according to a conventional method of preparing an injection, thereby preparing an injection with the above components per ampule (2 ml) according to a conventional method of preparing an injection.

### 1-5. Preparation of Liquid

According to a conventional method for preparing a liquid, 20 mg of the mixed extract of Achyranthes japonica and Glycyrrhiza uralensis prepared in any one of Examples 1.3 to 1.8, 10 g of isomerized sugar, and 5 g of mannitol were added to purified water to dissolve the mixed extract therein, a suitable amount of lemon flavor was added thereto, and the above components were mixed. Then, purified water was further added to be adjusted to the total 100 mL, filled in a brown bottle, and sterilized to prepare a liquid.

### Preparation Example 2. Preparation of Food Composition

### 2-1. Preparation of Health Functional Food

A health functional food was prepared by mixing 100 mg of the mixed extract of Achyranthes japonica and Glycyrrhiza uralensis prepared in any one of Examples 1.3 to 1.8, an appropriate amount of a vitamin mixture, 70 g of vitamin A acetate, 1.0 mg of vitamin E, 0.13 mg of vitamin B1, 0.15 mg of vitamin B2, 0.5 mg of vitamin B6, 0.2 g of vitamin B12, 10 mg of vitamin C, 10 g of biotin, 1.7 mg of nicotinic acid amide, 50 g of folic acid, 0.5 mg of calcium pantothenate, an appropriate amount of an inorganic mixture, 1.75 mg of ferrous sulfate, 0.82 mg of zinc oxide, 25.3 mg of magnesium carbonate, 15 mg of potassium phosphate monobasic, 55 mg of potassium phosphate dibasic, 90 mg of potassium citrate, 100 mg of calcium carbonate, and 24.8 mg of magnesium chloride, and then preparing granules according to a conventional method. In this case, the composition ratio of the vitamin and mineral mixture was made by mixing components relatively suitable for the health functional food in the preferred embodiment, but the mixing ratio may be arbitrarily modified.

### 2-2. Preparation of Health Functional Beverage

According to a conventional method of preparing a health beverage, 100 mg of the mixed extract of Achyranthes japonica and Glycyrrhiza uralensis prepared in any one of Examples 1.3 to 1.8, 15 g of vitamin C, 100 g of vitamin E (powder), 19.75 g of ferrous lactate, 3.5 g of zinc oxide, 3.5 g of nicotinic acid amide, 0.2 g of vitamin A, 0.25 g of vitamin B1, 0.3 g of vitamin B2, and a fixed amount of water were mixed, stirred and heated at 85° C. for about one hour, after which the resulting solution was filtered, obtained in a sterilized 2 L container, sealed, sterilized, and stored in a refrigerated state to prepare a health beverage. In this case, although the composition ratio is prepared by mixing ingredients relatively suitable for the favorite beverage in a preferred embodiment, the mixing ratio may be arbitrarily modified according to the preference of local and ethnic groups such as a class for needs, country for needs, and purpose for use.

From the above description, those skilled in the art to which the present invention pertains will understand that the present invention may be implemented in other specific forms without changing the technical spirit or essential features thereof. In this regard, it needs to be understood that the embodiments described above are exemplary and not restrictive in all aspects. The scope of the present invention needs to be construed as including all changes or modifications derived from the meaning and scope of the claims to be described later and the equivalent concept thereof in the scope of the present invention rather than the detailed description.

1. A food composition for preventing or ameliorating eye diseases, comprising Achyranthes japonica, Glycyrrhiza uralensis, or a mixed extract thereof.

2. The food composition of claim 1, wherein the extract is obtained by extracting with a solvent selected from the group consisting of water, a straight or branched-chain alcohol having one to six carbon atoms, an organic solvent, and a mixed solvent thereof.

3. The food composition of claim 1, wherein the extract is obtained by extracting with an aqueous ethanol solution.

4. The food composition of claim 3, wherein the aqueous ethanol solution is 25 to 55% aqueous ethanol solution.

5. The food composition of claim 1, wherein the mixed extract is obtained by mixing Achyranthes japonica and Glycyrrhiza uralensis in a weight ratio of 1:1 to 1:9.

6. The food composition of claim 1, wherein the extract is included in an amount of 0.001 to 90 wt % based on the total weight of the food composition.

7. The food composition of claim 1, wherein the food composition is prepared as powders, granules, tablets, capsules, syrups, or beverages.

8. The food composition of claim 1, wherein the eye diseases include macular degeneration, glaucoma, diabetic retinopathy, retinal edema, an epiretinal membrane, a retinal break, cataract, dry eye, superficial keratitis, or retinal and corneal abnormalities.

9. A pharmaceutical composition for preventing or treating eye diseases, comprising Achyranthes japonica, Glycyrrhiza uralensis, or a mixed extract thereof.

10. A quasi-drug composition for preventing or ameliorating eye diseases, comprising Achyranthes japonica, Glycyrrhiza uralensis, or a mixed extract thereof.

11. A feed composition for preventing or ameliorating eye diseases, comprising Achyranthes japonica, Glycyrrhiza uralensis, or a mixed extract thereof.

12. A method for preventing or treating eye diseases, the method comprising administering a pharmaceutical composition including Achyranthes japonica, Glycyrrhiza uralensis, or a mixed extract thereof into an individual.
